(19) United States
(12) Patent Application Publication
(10) Pub. No.: US 2007/0196342 A1
(43) Pub. Date: Aug. 23, 2007

(54) Meniscal implant of hyaluronic acid derivatives for treatment of meniscal defects

(76) Inventors: Khalid K. Sadozai, Shrewsbury, MA (US); Carol A. Toth, Webster, NY (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/638,087

(22) Filed: Dec. 13, 2006

Related U.S. Application Data
(60) Provisional application No. 60/751,237, filed on Dec. 14, 2005.
(60) Provisional application No. 60/751,381, filed on Dec. 14, 2005.
(60) Provisional application No. 60/751,414, filed on Dec. 14, 2005.

(51) Int. Cl. A61K 35/12; A61K 38/18; A61K 31/728; A61K 9/70

(52) U.S. Cl. 424/93.7; 514/12; 514/54; 424/443; 536/53

(57) ABSTRACT

A composite for treating an articular defect includes a hyaluronic acid derivative; and at least one member of the group consisting of a cell, a cellular growth factor and a cellular differentiation factor, which is impregnated in, or coupled to, the hyaluronic acid derivative. In one embodiment, carboxyl functionalities of the hyaluronic acid derivative are each independently derivatized to include an N-acylurea or O-acyl isourea, or both N-acylurea and O-acyl isourea. In another embodiment, the hyaluronic acid derivative is prepared by reacting an uncrosslinked hyaluronic acid with a biscarbodimide in the presence of a pH buffer in a range of between about 4 and about 8. The composite can be used for regenerating or stimulating regeneration of meniscal tissues in a subject in need thereof.

### INCORPORATION BY REFERENCE

This application claims the benefit of U.S. Provisional Application Nos. 60/751,237; 60/751,381; and 60/751,414, all of which were filed Dec. 14, 2005. The entire teachings of the above-mentioned applications are incorporated herein by reference.

### BACKGROUND OF THE INVENTION

The medial and lateral menisci are a pair of biconcave, generally C-shaped wedges of fibrocartilage structures in the knee joint. The major meniscal functions are to distribute stress across the knee during weight bearing, provide shock absorption, serve as secondary joint stabilizers, provide articular cartilage nutrition and lubrication, facilitate joint gliding, prevent hyperextension, and protect the joint margins. Typically, when the meniscus is damaged, the torn piece begins to move inside the joint in an abnormal way. Because the space between the bones of the joint is very small, as the torn piece of meniscal tissue moves abnormally, it may become caught between the bones of the joint. When this happens, the knee becomes painful, swollen and difficult to move.

Traditional treatments of injured or diseased meniscus have been removal of the meniscus (meniscectomy) and surgical repair of the meniscus. The traditional treatments of injured or diseased meniscus have some problems. For example, meniscectomy is often followed by degenerative changes within the knee joint. Replacement of injured meniscus in an otherwise healthy knee joint may prevent arthritic changes and may stabilize the joint. However, replacement of meniscal tissues with structures consisting of permanent artificial materials has generally been unsuccessful. This lack of success is partly due to the fact that opposing articular cartilage of human and animal knee joints is fragile. The articular cartilage in the knee joint generally does not withstand abrasive interfaces or compliance variances from normal, which may result from implanted artificial prosthetic meniscus. In addition, joint forces are multiples of body weight which are typically encountered over a million cycles per year in the case of the knee and hip. Thus far, permanent artificial meniscus has not been composed of materials having natural meniscal properties, nor has it been able to be positioned securely enough to withstand such routine forces.

Therefore, there is a need to develop methods for treating articular defects, such as meniscal defects, that can overcome one or more of the aforementioned problems.

### SUMMARY OF THE INVENTION

The invention generally is directed to a composite for treating an articular defect and the use of the composite for a meniscal augmentation device and methods for treating such a defect.

In one aspect, the invention is directed to a composite for treating an articular defect, such as a meniscal defect. The composite includes a hyaluronic acid derivative, and at least one member of the group consisting of a cell, a cellular growth factor and a cellular differentiation factor. The cell, cellular growth factor or cellular differentiation factor is impregnated in, or coupled to, the hyaluronic acid derivative. In one embodiment, carboxyl functionalities of the hyaluronic acid derivative are each independently derivatized to include an N-acylurea or O-acyl isourea, or both N-acylurea and O-acyl isourea. In another embodiment, the hyaluronic acid derivative is prepared by reacting an uncrosslinked hyaluronic acid with a biscarbodimide in the presence of a pH buffer in a range of between about 4 and about 8.

In another aspect, the invention is directed to a meniscal augmentation device having a shape of a meniscal defect in meniscus. The meniscal augmentation device includes a hyaluronic acid derivative, and at least one member of the group consisting of a cell, a cellular growth factor and a cellular differentiation factor, which is impregnated in, or coupled to, the hyaluronic acid derivative. Preferably, the cell, cellular growth factor and cellular differentiation factor include at least one member selected from the group consisting of mesencymal stem cells, periosteal cells, synoviocytes, fibrochondrocytes, osteochondrocytes, chondrocytes, TGFb supergene family members, tissue growth hormones, encoding genes thereof, and synthetic peptide analogues thereof. The hyaluronic acid derivative is as described above for the composite for treating an articular defect.

The present invention also provides a method of regenerating or stimulating regeneration of meniscus tissues in a subject. The method includes implanting a composite at a site of the meniscus defect. The composite includes a hyaluronic acid derivative, and at least one member of the group consisting of a cell, a cellular growth factor and a cellular differentiation factor, which is impregnated in, or coupled to, the hyaluronic acid derivative. The hyaluronic acid derivative is as described above for the composite for treating an articular defect.

The composite and meniscal augmentation device of the invention can treat or enhance the natural repair of meniscal defects. For example, the composite and meniscal augmentation device employing subject's own cells can provide structural support and molecular cuing to stimulate regeneration of meniscus tissue using the subject's own healing process. The hyaluronic acid derivative will be absorbed by the body while the tissue regeneration takes place. Thus, there is no need to remove the hyaluronic acid derivative from the subject after the regenerated tissues restore their function, leaving no artificial materials at the treatment site. In addition, the composite and meniscal augmentation device can be formed in the shape of the meniscus or in the form of a sheet or film that can be cut, trimmed and contoured to fit correctly and completely fill the meniscus defect, enhancing efficacy of the composite and meniscal augmentation device.

### BRIEF DESCRIPTION OF THE DRAWINGS

FIGS. 1A and 1B are scanning electron microscopy (SEM) images of a composite of the invention, including a freeze-dried crosslinked hyaluronic acid (HA) sponges, at two different magnifications.

FIGS. 2A and 2B are scanning electron microscopy (SEM) images of a composite of the invention, including a freeze-dried crosslinked hyaluronic acid (HA) sponges, at two different magnifications.

FIG. 3 is a cross sectional view of the composite of FIGS. 2A-2B, showing interconnected structural support that can provide cues for ingrowth of cells, cellular growth factors or cellular differentiation factors for tissue regeneration.

### DETAILED DESCRIPTION OF THE INVENTION

The foregoing and other objects, features and advantages of the invention will be apparent from the following more particular description of preferred embodiments of the invention, as illustrated in the accompanying drawings. The drawings are not necessarily to scale, emphasis instead being placed upon illustrating the principles of the invention.

As used herein, the term “hyaluronic acid derivative” means hyaluronic acid derivatized in that carboxyl functionalities of the hyaluronic acid (HA) (a portion or all) are each independently derivatized to include an N-acylurea or O-acyl isourea, or both N-acylurea and O-acyl isourea. As used herein, hyaluronic acid, and any of its salts which are often referred to as “hyaluronan” (e.g., sodium, potassium, magnesium, calcium or ammonium salts) are represented by the term “HA.” Typically, HA comprises disaccharide units of D-glucuronic acid (GlcUA) and N-acetyl-D-glucosamine (GlcNAc), which are alternately linked, forming a linear polymer.

N-acylurea and O-acyl isourea derivatives for the invention are as shown in the bracketed fragments in the following structural formulas (I) and (II):

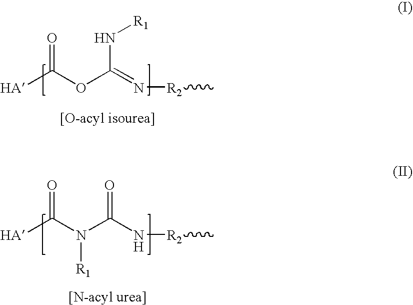

In structural formulas (I) and (II), each R₁ can be the same or different. Each R₁ is selected from the group consisting of hydrogen; substituted or unsubstituted hydrocarbyl groups (linear or branched, or cyclic or acyclic) optionally interrupted by one or more heteroatoms; substituted or unsubstituted alkoxy; substituted or unsubstituted aryloxy; and substituted or unsubstituted aralkyloxy. Examples of substituted or unsubstituted hydrocarbyl groups (linear or branched, or cyclic or acyclic) optionally interrupted by one or more heteroatoms include optionally substituted aliphatic groups (e.g., alkyl, alkenyl, alkynyl, cycloalkyl, cycloalkenyl, cycloalkynyl and cycloaliphaticalkyl); optionally substituted aryl groups (including heteroaryl groups); optionally substituted aliphatic groups interrupted by one or more heteroatoms (e.g., heterocyclyl, cycloaliphaticalkyl and heterocyclylalkyl); and optionally substituted, partially aromatic and partially aliphatic groups (e.g., aralkyl and heteroaralkyl). Suitable optional substituents are those that do not substantially interfere with the properties of the resulting crosslinked HA composition. Suitable substituents for carbon atoms of hydrocarbyl groups include —OH, halogens (—Br, —Cl, —I, —F), —OR⁸, —O—COR^{a}, —COR^{a}, —CN, —NCS, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{b}COR^{a}, —NHCONH₂, —NHCONR^{a}H, —NHCON(R^{a}R^{b}), —NR^{b}CONH₂, —NR^{b}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —SH, —SR^{a}, —S(O)R^{a}, and —S(O)₂R^{a}. In addition, an alkyl, alkylene, alkenyl or alkenylene group can be substituted with substituted or unsubstituted aryl group to form, for example, an aralkyl group such as benzyl. Similarly, aryl groups can be substituted with a substituted or unsubstituted alkyl or alkenyl group.

R^{a}-R^{d} are each independently an alkyl group, aryl group, including heteroaryl group, non-aromatic heterocyclic group or —N(R^{a}R^{b}), taken together, form a substituted or unsubstituted non-aromatic heterocyclic group. The alkyl, aromatic and non-aromatic heterocyclic group represented by R^{a}-R^{d} and the non-aromatic heterocyclic group represented by —N(R^{a}R^{b}) can optionally be substituted.

In other embodiments, R¹ is an optionally substituted aliphatic group (cyclic or acyclic, or linear or branched). More preferably, R¹ is an alkyl group, such as C1-C6 alkyl (e.g., methyl, ethyl, propyl, butyl, 2-propyl, tert-butyl, and the like). Preferably, each R₁ is ethyl.

Each R₂ is independently a substituted or unsubstituted linking group including one or more of hydrocarbylene groups (cyclic or acyclic, or linear or branched) optionally interrupted by one or more heteroatoms. Examples include optionally substituted aliphatic groups (e.g., alkylene, alkenylene, alkynylene, cycloalkylene, cycloalkenylene, cycloalkynylene and cycloaliphaticalkylene); optionally substituted arylene (including heteroaryl groups); optionally substituted aliphatic groups interrupted by one or more heteroatoms (e.g., heterocyclylene, cycloaliphaticalkylene and heterocyclylalkylene); and optionally substituted, partially aromatic and partially aliphatic groups (e.g., aralkylene and heteroaralkylene). Suitable optional substituents are as those described above for R₁.

In some embodiments, R₂ includes or is interrupted by other groups, e.g, carbonyl, amide, oxy, sulfide, disulfide, and the like. In other embodiments, R₂ is a cycloaliphatic, arylene, heteroarylene, or heterocyclylene group. In still other embodiments, R₂ is 1,6-hexamethylene, octamethylene, decamethylene, dodecamethylene, PEG, —CH₂CH₂—S—S—CH₂CH₂—, para-phenylene-S—S-para-phenylene, meta-phenylene-S—S-meta-phenylene, ortho-phenylene-S—S-ortho-phenylene, ortho-phenylene, meta-phenylene or para-phenylene. More preferably, R₂ is phenylene. Preferably, R₂ is para-phenylene.

In one embodiment, the wavy line connected to R₂ in structural formulas (I) and (II) represents hydrogen, substituted or unsubstituted hydrocarbyl groups (linear or branched, or cyclic or acyclic) optionally interrupted by one or more heteroatoms; alkoxy; aryloxy; or aralkyloxy, as described for R₁. In another embodiment, the wavy line connected to R₂ in structural formulas (I) and (II) represents optionally substituted N-acyl urea group or O-acyl isourea group, as shown below in structural formulas VI-VIII.

In general, the modified HA derivative is prepared by reacting hyaluronic acid, or a salt thereof, with a carbodiimide, preferably a multifunctional carbodiimide, such as a biscarbodiimide, in the absence of a nucleophile or a polyanionic polysaccharide other than HA, to form an N-acylurea or O-acyl isourea.

Examples of suitable carbodiimides in the invention include a monocarbodiimide and a multifunctional carbodiimide, such as a biscarbodiimide. The monocarbodiimide has the formula:
R₃—N═C═N—R₄ (III)
wherein R₃ and R₄ are each independently as described above for R₁ (e.g., hydrocarbyl, substituted-hydrocarbyl, alkoxy, aryloxy or alkaryloxy). Examples of suitable monocarbodiimides include: 1-ethyl-3-(3-dimethylaminopropyl)-carbodiimide hydrochloride (EDC); 1-cyclohexyl-3-(2-morpholinoethyl)carbodiimide metho-p-toluenesulfonate (CMC); 1-(3-(dimethylamino)propyl)-3-ethylcarbodiimide methiodide (EAC); 1,3-dicyclohexylcarbodiimide (DCC); and 1-benzyl-3-(3-dimethylaminopropyl)carbodiimide hydrochloride (BDC).

Examples of suitable biscarbodiimides may be represented by those difunctional compounds having the formula:
R₁—N═C═N—R₂—N═C═N—R₁ (IV).
Each R₁ can be different or the same. R₁ and R₂ are each independently as described above. Suitable specific examples of biscarbodiimides include 1,6-hexamethylene bis(ethylcarbodiimide), 1,8-octamethylene bis(ethylcarbodiimide), 1,10 decamethylene bis(ethylcarbodiimide), 1,12 dodecamethylene bis(ethylcarbodiimide), PEG-bis(propyl(ethylcarbodiimide)), 2,2′-dithio-bis(ethyl(ethylcarbodiimde)), 1,1′-dithio-ortho-phenylene-bis(ethylcarbodiimide), 1,1′-dithio-para-phenylene-bis(ethylcarbodiimide), and 1,1′-dithio-meta-phenylene bis(ethylcarbodiimide). In a preferred embodiment, the biscarbodiimide is para-phenylene-bis(ethylcarbodiimide). Methods of preparing biscarbodiimides are described, for example, in U.S. Pat. Nos. 6,013,679; 2,946,819; 3,231,610; 3,502,722; 3,644,456; 3,972,933; 4,014,935; 4,066,629; 4,085,140; 4,096,334; 4,137,386, 6,548,081, and 6,620,927 the teachings of which are incorporated herein by reference in their entireties.

In a preferred embodiment, the HA derivative is crosslinked. In a more preferred embodiment, the HA derivative is at least about 1% by mole crosslinked, and the HA derivative includes at least one crosslink, e.g., the linking group connecting through a group U at each end to a HA′ molecule, as shown in the following structural formula:
HA′-U—R₂—U—HA′ (V).
Each HA′ in the preceding formula can be different or the same HA′ molecule, e.g., the crosslink can be an intermolecular or intramolecular crosslink. Each U can be the same or different and is an optionally substituted N-acyl urea or O-acyl isourea. As used herein, the term “at least about 1% by mole crosslinked” means that HAs are crosslinked with each other via derivatized carboxyl functionalities of the HAs, such as O-acylisoureas or N-acylureas, wherein the derivatized carboxyl functionalities are at least about 1% by mole of the total carboxyl functionalities of the individual HA.

In an even more preferred embodiment, the N-acylurea or O-acylisourea results from crosslinking with the multifunctional carbodiimide. Alternatively, a monocarbodiimide may be employed in combination with a multifunctional carbodiimide. Suitable examples of monocarbodiimides and multifunctional carbodiimides are as described above. Use of a multifunctional carbodiimide to prepare the modified HA derivative causes crosslinking of the hyaluronic acid. For example, use of a biscarbodiimide results in a crosslinking between COOH groups present in the repeating disaccharide unit of hyaluronic acid, since the biscarbodiimide is difunctional. The COOH group may be present in the same polymer chain, resulting in an intramolecular crosslinked product, or present on two different polymer chains, resulting in an intermolecular crosslinked product.

The reaction of HA with a biscarbodiimide rather than a monocarbodiimide does not change the mechanism of reaction, but can cause the product to be crosslinked.

The reaction of HA with a biscarbodiimide crosslinking reagent, in the presence of an available proton, is believed to comprise protonation in the first step. The acid anion can then attach to the carbon atom of the cation formed, resulting in the formation of an O-acyl isourea intermediate. The acyl group in the intermediate can migrate from the oxygen atom to a nitrogen atom to produce a N-acyl isourea derivative of the HA. It is believed that the O-to-N migration can be incomplete, resulting in a product reaction mixture that can include both the N-acyl urea and the O-acyl isourea. Thus, a crosslink resulting from reaction of a biscarbodiimide with the uncrosslinked HA precursor typically can contain two O-acyl isoureas connected through R₂, as represented in the following structural formula (VI): 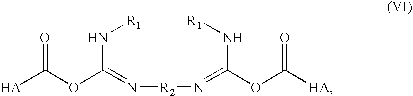
or an O-acyl isourea and an N-acyl urea connected through R₂, as represented in the following structural formula (VII): 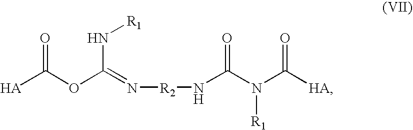
or two N-acyl ureas connected through R₂, as represented in the following structural formula (VIII):

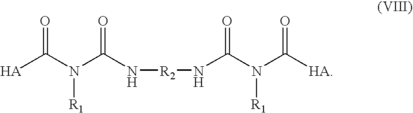

The mixed products can be used separately or together to prepare the compositions according to embodiments of the invention.

The term “hydrocarbyl,” as used herein, means a monovalent moiety obtained upon removal of a hydrogen atom from a parent hydrocarbon. As used herein, hydrocarbylene groups are divalent hydrocarbons. Typically, hydrocarbyl and hydrocarbylene groups contain 1-25 carbon atoms, 1-12 carbon atoms or 1-6 carbon atoms. Hydrocarbyl and hydrocarbylene groups can be independently substituted or unsubstituted, cyclic or acyclic, branched or unbranched, and saturated or unsaturated. Optionally, hydrocarbyl and hydrocarbylene groups independently can be interrupted by one or more hetero atoms (e.g., oxygen, sulfur and nitrogen). Examples of hydrocarbyl groups include aliphatic and aryl groups. Substituted hydrocarbyl and hydrocarbylene groups can independently have more than one substituent.

The term “substitutent,” as used herein, means a chemical group which replaces a hydrogen atom of a molecule. Representative of such groups are halogen (e.g., —F, —Cl, —Br, —I), amino, nitro, cyano, —OH, alkoxy, alkyl, alkenyl, alkynyl, aryl, haloalkoxy, haloalkyl, haloalkenyl, haloalkynyl, alkyl amino, haloalkyl amino, aryl amido, sulfamido, sulfate, sulfonate, phosphate, phosphino, phosphonate, carboxylate, carboxamido, and the like.

An “alkyl” group, as used herein, is a saturated aliphatic group. The alkyl group can be straight chained or branched, or cyclic or acyclic. Typically, an alkyl group has 1-25 carbon atoms. Examples of alkyl groups include methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonodecyl, eicosyl, heneicosyl, docosyl, tricosyl, tetracosyl, pentacosyl, and the isomeric forms thereof. An alkyl group may be substituted with one or more substituents independently selected for each position.

An “alkylene” group, as used herein, is a saturated aliphatic group that is bonded to two other groups each through a single covalent bond. The alkylene group can be straight chained or branched, or cyclic or acyclic. Typically, an alkylene group has 1-25 carbon atoms. Examples of alkylene groups include methylene, ethylene, propylene, butylene, pentylene, hexylene, heptylene, octylene, 1,6-hexamethylene, 1,8-octamethylene, 1,10-decamethylene, 1,12-dodecamethylene and the isomeric forms thereof. An alkylene group may be substituted with one or more substituents independently selected for each position.

As used herein, an “alkenyl” group is an aliphatic group that contains a double bond. Typically, an alkenyl group has 2 to 25 carbon atoms. Examples include vinyl, allyl, butenyl, pentenyl, hexenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, docosenyl, tricosenyl, tetracosenyl, pentacosenyl, and isomeric forms thereof.

As used herein, an “alkenylene” group is an aliphatic group that contains a double bond. Typically, an alkenylene group has 2 to 25 carbon atoms. Examples include butenylene, pentenylene, hexenylene, octenylene, nonenylene and isomeric forms thereof.

As used herein, an “alkynyl” group is an aliphatic group that contains a triple bond. Typically, an alkynyl group has 2 to 25 carbon atoms. Examples include vinyl, allyl, butynyl, pentynyl, hexynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, heneicosynyl, docosynyl, tricosynyl, tetracosynyl, pentacosynyl, and isomeric forms thereof.

As used herein, an “alkynylene” group is an aliphatic group that contains a triple bond. Typically, an alkynylene group has 2 to 25 carbon atoms. Examples include vinylene, allylene, butynylene, pentynylene, hexynylene, octynylene and isomeric forms thereof.

The term “aryl” as used herein refers to an aromatic ring (including heteroaromatic ring). Particularly, an aryl group that includes one or more heteroatoms is herein referred to “heteroaryl.” Examples of aryl groups include phenyl, tolyl, xylyl, naphthyl, biphenylyl, triphenylyl, and heteroaryl, such as pyrrolyl, thienyl, furanyl, pyridinyl, oxazolyl, isooxazolyl, thiazolyl, isothiazolyl and quinolinyl. An aryl group may be substituted with one or more substituents independently selected for each position.

The term “arylene” as used herein refers to an aryl ring in a molecule that are bonded to two other groups each through a single covalent bond from two of its ring atoms. Particularly, an arylene group that includes one or more heteroatoms is herein referred to “heteroarylene.” Examples of arylene groups include phenylene [—(C₆H₄)—], such as meta-phenylene and para-phenylene; and heteroarylene groups, such as pyridylene [—(C₅H₃N)—]; and furanylene [—(C₄H₂O)—]. An arylene group may be substituted with one or more substituents independently selected for each position.

An alkyl, alkylene, alkenyl, alkenylene group, alkynyl or alkynylene can be optionally substituted with substituted or unsubstituted aryl group to form, for example, an aralkyl group (e.g. benzyl), or aralylene (e.g. —CH₂—(C₆H₄)— or —CH═CH₂—(C₆H₄)—). Similarly, aryl or arylene groups can be optionally substituted with a substituted or unsubstituted alkyl, alkenyl or alkynyl group.

The term “heterocyclyl” refers to a cycloalkyl group wherein one or more ring carbon atoms are replaced with a heteroatom, e.g., aziridyl, azetidyl, pyrrolidyl, piperidyl, thiiranyl, thietanyl, tetrahydrothiophenyl, tetrahydrothiopyranyl, oxiranyl, oxetanyl, tetrahydrofuranyl, tetrahydropyranyl, morpholinyl, and the like.

The term “heterocyclylene” refers to a cycloalkylene group wherein one or more ring carbon atoms are replaced with a heteroatom, e.g., 2,5-tetrahydrofuranylene.

An alkoxy group is an alkyl group connected through an oxygen atom, e.g., methoxy, ethoxy, propoxy and the like.

An aryloxy group is an aryl group connected through an oxygen atom, e.g., phenoxy and the like.

An aralkyloxy group is an aralkyl group connected through an oxygen atom, e.g., benzyl oxy and the like.

In one embodiment, the modified HA derivative is at least about 1% by mole crosslinked. The crosslinked HA gel can be water-soluble or substantially water-insoluble.

In another embodiment, at least about 1% by mole, such as at least about 2% by mole, at least about 5% by mole, or between about 1% by mole and about 20% by mole, of the carboxyl functionalities of the modified hyaluronic acid are derivatized. In yet another embodiment, at least about 25% by mole, such as between about 25% by mole and about 75% by mole, of the derivatized functionalities are O-acylisoureas and/or N-acylureas. In yet another embodiment, the carboxyl functionalities of the modified hyaluronic acid are derivatized, and the derivatized carboxyl functionalities result from crosslinking of HAs with a multifunctional carbodiimide described above, preferably biscarbodiimide. Conditions for such crosslinkings are known in the art, for example, in U.S. Pat. No. 6,548,081, the entire teachings of which are incorporated herein by reference.

The steps required to make a biocompatible HA derivative include providing a sample of HA or a salt thereof, such as sodium hyaluronate. HA from any of a variety of sources, including HA extracted from animal tissues or harvested as a product of bacterial fermentation, can be used as a starting material. Alternatively, the HA used to make the composites of this invention can be produced in commercial quantities by bioprocess technology, as described, for example, in Nimrod et al., PCT Publication No. WO 86/04355.

In one example, the sample of HA or its salt is dissolved in water to make an aqueous solution. In a particular example, the concentration of HA in this first aqueous solution is in the range of between about 0.1% and 5% weight/weight (“w/w”), that is, Img/ml solution to 50 mg/ml solution. In another particular example, the reactions are carried out with a range of about between about 0.4% and 0.6% weight/weight, or 4 to 6 mg of hyaluronic acid per milliliter. The precise concentration used will vary depending on the molecular weight of the HA. At significantly lower concentrations, the reactions are slower and less effective. At significantly higher HA concentrations, the end product may be difficult to handle due to the increase in viscosity. One skilled in the art will be able to determine, with no more than routine experimentation, an acceptable concentration of HA to be used for a particular embodiment. Examples of various acceptable concentrations of HA are described in U.S. Pat. No. 5,356,883, to Kuo et al., the teachings of which are incorporated herein by reference in their entirety.

The pH of the HA solution is then adjusted by the addition of a suitable acid or a suitable pH buffer known in the art, so that the aqueous HA solution is acidic, preferably having a pH of about between 4.0 and 8.0, such as about between 4.0 and about 6.0 or between about pH 4.75 and about pH 5.5. The pH buffer can include any buffer agent known to one skilled in the art, e.g., 2-(N-morpholino)ethanesulfonic acid (MES); 2,2-bis(hydroxymethyl)-2,2′,2″-nitrotriethanol; succinate/succinic acid; KH₂PO₄; N-tris(hydroxymethyl-2-aminoethanesulfonic acid; triethanolamine; diethylbarbituate; tris(hydroxymethyl)aminoethane; N-tris(hydroxy)methylglycine; and N,N-bis(2-hydroxyethyl)glycine. The buffer agent can be employed with an additional acid or base, e.g., 2-(N-morpholino)ethanesulfonic acid with NaOH; 2,2-bis(hydroxymethyl)-2,2′,2″-nitrotriethanol with HCl; succinate with succinic acid; KH₂PO₄ with borax; N-tris(hydroxymethyl-2-aminoethanesulfonic acid with NaOH; triethanolamine with HCl; diethylbarbituate with HCl; tris(hydroxymethyl)aminoethane with HCl; N-tris(hydroxy)methylglycine with HCl; and N,N-bis(2-hydroxyethyl)glycine with HCl. Preferably, the buffer includes 2-(N-morpholino)ethanesulfonic acid and NaOH.

Once the pH of the aqueous HA solution has been adjusted, the carbodiimide can be added. Generally an excess of the stoichometric proportion of carbodiimide is advantageous to promote the desired reaction. Preferably the molar equivalent ratio of the carbodiimide to HA is equal to or greater than about 5%.

In one example, the pH of the aqueous HA solution is adjusted by the addition of a suitable acid, such as an HCl solution. Preferably, the carbodiimide is dissolved in an appropriate water-mixable solvent and added drop-wise. In this example, as the carbodiimide and the HA are mixed, the pH of the solution generally increases. Films and gels with various desired physical properties can be obtained by simply allowing the pH to rise as the reaction proceeds. However, the reaction is monitored by a pH meter, and HCl may be added to maintain the pH of the reaction mixture, for example, about between 4.0 and 8.0, such as about between 4.0 and about 6.0 or between about pH 4.75 and about pH 5.5. The reaction is then allowed to proceed at room temperature for about two hours. The reaction may be directed to favor the formation of the N-acylurea derivatives by increasing the pH with a suitable aqueous base. The progress of the reactions described above may be followed by monitoring the pH. When the pH is stabilized, the reactions are substantially complete.

In another example, the carbodiimide, such as biscarbodiimide, is reacted with the HA in the presence of a suitable pH buffer, wherein the buffer is at a pH between about 4 and about 8. Suitable examples of pH buffer agents are as described above. Typically, the buffer agent is mixed in aqueous media, in a concentration between about 5 mM (millimolar) and about 250 mM (e.g., about 75 mM). Typically, the HA is mixed in aqueous media, e.g., the pH buffer solution, in a concentration between about 1 mM (millimolar) and about 100 mM (e.g., about 37 mM). The particular concentration employed can vary depending on the molecular weight of the HA.

The carbodiimide can be combined with the HA solution alone, or more typically as a solution in a water-miscible organic solvent, e.g., acetone, methyl ethyl ketone, dimethyformamide, dimethyl sulfoxide, methanol, ethanol, 2-propanol, acetonitrile, tetrahydrofuran, N-methyl pyrrolidone, and the like. When a biscarbodiimide is utilized, typically, the solvent is acetone, and the biscarbodiimide is at a concentration of between about 0.1 mg/mL and about 100 mg/mL. The HA and the carbodiimide, such as carbodiimide, can be combined in any molar equivalent ratio, e.g., between about 1% and about 200%, typically between about 2% and about 30%. The reaction can be carried out at a temperature range of between about 0° C. and about 60° C., typically between 25-30° C.

Crosslinked HA can be formed by reacting uncrosslinked HA with a crosslinking agent, such as a biscarbodiimide as described above, under suitable reaction conditions by methods known in the art, for example, ?U.S. patent application Ser. Nos. 10/743,557, U.S. Pat. Nos. 5,356,883, 5,502,081, 6,013,679, 6,537,979, and 6,548,081, the entire teachings of which are incorporated herein by reference. The uncrosslinked HA used as a precursor for the crosslinking typically has typically an average molecular weight range of from between about 6×10⁴ to about 8×10⁶ Daltons, or 150 to 20,000 disaccharide repeat units. Uncrosslinked HA having lower or higher molecular weights than these can also be used in the invention.

The reaction conditions for HA crosslinking with a biscarbodiimide are similar to those used for HA-monocarbodiimide coupling reactions. Advantageously, the crosslinking reactions are carried out with (1) an increase of the HA concentration in the reaction mixture, and/or (2) a decrease of the biscarbodiimide concentration in the addition solution. This creates a condition favorable to intermolecular crosslinking versus intramolecular crosslinking.

At the conclusion of the reactions described above, the desired HA derivative may be separated from the reaction mixtures by conventional methods of precipitation, washing and re-precipitation. The completeness of the reaction, the nature of the products and the extent of chemical modification can be determined by, for example, proton NMR, or by studying the resistance to enzymatic hydrolysis or studying other changes in the physical or chemical behavior of the product.

If a colored product is desired, a solution of a biocompatible dye or stain, e.g., Coomassie™ Brilliant Blue R-250, can be admixed to the reaction mixtures described above. The resulting product will have a blue color which makes the gel, film or sponge easy to see when it is handled during surgery and when it is in place.

When the reaction is complete, sodium chloride is typically added to the reaction mixture to adjust the sodium chloride concentration to 1M. Ethanol is added to form a precipitate of chemically-modified, HA derivative. The precipitate is separated from the solution, washed, and dried by vacuum. The freeze dried material can be washed with appropriate solvents to remove contaminants of the reaction and dried and then sterilized by ethylene oxide (EtO) sterilization or sterilization by gamma irradiation before loading the cells and implanting them into mammals.

To make a gel of the HA derivative, the precipitate is re-suspended in water and stirred in a cold room. The gel of the HA derivative is a hydrogel. The term “hydrogel” is defined herein to mean a macromolecular network swollen in water or biological fluids. The degree of hydration is dependent on the degree of crosslinking.

To make a sponge, the precipitate is then re-suspended in water, poured into a mold having a desired shape, and, preferably, dried, such as by air-drying, freeze-drying or heat-drying. A film may be prepared by further drying the gel. Alternatively, a film can be formed by compressing a gel under conditions that permit water to escape, as, for example, by compressing the gel between two surfaces, at least one of which is porous. See, for example, Malson et al., U.S. Pat. No. 4,772,419, the teachings of which are incorporated herein by reference in their entirety.

The composites of the invention can include the modified HA derivative described above without biocompatible, biodegradable supports (e.g., polymers) other than the modified HA derivative. Preferably, in this embodiment, the modified HA derivative is a highly-crosslinked HA, such as at least about 75% crosslinked HA.

Alternatively, the composites of the invention can include the modified HA derivative described above and one or more biocompatible, biodegradable supports (e.g., polymers) other than the modified HA derivative. In this embodiment, the modified HA is at the support(s). Preferably, in this embodiment, the modified HA derivative is a crosslinked HA of low degree of crosslinking, such as less than about 20%, such as about 5% or about 18%, or between about 1% and about 10% .

As used herein, a “biocompatible” support is one that has no medically unacceptable toxic or injurious effects on biological function. As used herein, a “biodegradable” support is one that is capable of being decomposed by natural biological processes.

Examples of the physical form of a suitable support include: a biocompatible, biodegradable matrix, sponge, film, sheet, thread, tube, non-woven fabric and cord. The biodegradable support may be formed from a material which is porous, and the pore sizes may be large enough so that when a layer of the hyaluronic acid (HA) derivative is spread on the support, the molecules of the HA derivative can partially or fully penetrate into the pores of the support to make an anchor. Examples of compositions to be used as a suitable support include: crosslinked alginates, gelatin, collagen, crosslinked collagen, collagen derivatives, such as, succinylated collagen or methylated collagen, crosslinked hyaluronic acid, chitosan, chitosan derivatives, such as, methylpyrrolidone-chitosan, cellulose and cellulose derivatives such as cellulose acetate or carboxymethyl cellulose, dextran derivatives such carboxymethyl dextran, starch and derivatives of starch such as hydroxyethyl starch, other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyoxanones and polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinylalcohol, polycasein, keratin, myosin, and fibrin.

A sample of highly crosslinked HA, for example at least about 75% by mole, crosslinked HA, may form a support for a sample of modified HA which is not highly crosslinked. One example of the highly-crosslinked HA is a thiol-containing, highly crosslinked HA (see U.S. Pat. No. 6,620,927, the entire teachings of which are incorporated herein by reference). The HA support can be made by, for example, pouring a mixture of uncrosslinked HA and a crosslinking agent, such as a biscarbodiimide having an intramolecular disulfide bond, into a mold and freeze dried in a desired shape.

The composite of the invention may be in a form of sponge, film, sheet, gel, thread, tube, non-woven fabrics, cords and meshes. In one embodiment, the composite is in the form of a sponge. In another embodiment, the composite is in the form of a sheet or film, preferably hydrophilic sheet or film. The hydrophilic sheet or film can be stacked together for stitching, for example, to correctly fit or fully fill a treatment site.

When the HA derivative is employed in combination with one or more biocompatible, biodegradable supports as described above, there are several ways in which the HA derivative (e.g., gel, film or sponge) can be immobilized at the support to make a composite device of this invention (see, for example, U.S. Pat. No. 6,548,081). For example, a layer of derivatized HA may be applied, either by soaking or dipping or spraying or spreading or by any other method of application, to at least one surface of a support to form a composite. A suitable support may be a matrix, sponge, film, sheet, gel, thread, tube, non-woven fabrics, cords and meshes, which may be porous. If the surface of the support is porous, the HA derivative will soak into the pores on the support surface. For example, porous beads may be soaked in the hyaluronic acid derivative for a sufficient period of time to allow the hyaluronic acid derivative to be absorbed and adsorbed by the pores of the beads. The composite is then dried under conditions that permit the escape of water from the composite.

In another embodiment, a composite sponge or film having hyaluronic acid derivative on both sides of the support is prepared by pouring the water-insoluble gel of derivatized HA prepared according to the procedure described above, into a first mold having the desired shape and depth, and spreading the gel in the first mold to form a first gel layer of even thickness. A suitable support may be a matrix, sponge, film, or particles such as beads made from another biocompatible material, for example collagen or gelatin. The support is spread on top of the evenly-spread first gel layer of derivatized HA. A second mold of the same size, shape and depth, is placed on the top of the support. Gel is poured into the second mold, and spread to form a second gel layer of even thickness in the second mold. In this manner, the polymer used as supporting matrix is sandwiched between the two layers of derivatized HA gel which are molded to the support. The composite is freeze-dried. The freeze-dried composite may be cut into specimens of the desired shape and size.

The composite of the invention can optionally include a material that enhances adherence of the composite to tissue. Materials that are suitable for enhancing adherence of the composite to tissue include fibrin, collagen, crosslinked collagen, and collagen derivatives, and any other polymers that include a peptide sequence having arginine (R), glycine (G), and aspartic acid (D), such as a peptide sequence consisting of arginine (R), glycine (G), and aspartic acid (D).

In a preferred embodiment, the composite of the invention is an implantable composite. More preferably, the implantable composite has interconnected pores of sizes that can provide molecular cuing for the impregnated or coupled cells, or a path for migration of the impregnated or coupled cellular growth factors or cellular differentiation factors. The interconnected pores of sizes can also provide molecular cuing for cells of a subject that are surrounding the treatment site (e.g., cartilage, bone or synovium) to migrate (or move) through. In a specific example, the implantable composite is freeze-dried and has interconnected pores of sizes that can provide molecular cuing for the impregnated or coupled cell to migrate through, or a path for migration of the impregnated or coupled cellular growth factor or cellular differentiation factor. The implantable composite can also provide molecular cuing for cells of the subject surrounding the treatment site to migrate through.

These composites can provide structural support and molecular cuing to stimulate regeneration of meniscal tissues by providing a mechanism for the delivery of, for example, cells to regenerate or promote regeneration of articular tissues, such as meniscus tissues. For example, the cells, cellular growth factors or cellular differentiation factors impregnated in or coupled to the hyaluronic acid derivatives can migrate into an articular defect site and regenerate articular tissues or repair the articular defect. The biocompatible, biodegradable support and the hyaluronic acid derivative will be absorbed by the body (e.g., by the regenerated tissues), while the tissue regeneration and remodeling takes place. After the biodegradation of the composite, the regenerated articular tissues, such as meniscus tissues, have the physical characteristics of natural articular tissues and assume their normal function.

Preferably, the composite of the invention has tear strength and tear propagation resistance and can be surgically sutured or anchored to stabilize it in a treatment site so that it does not move during the healing and regeneration process.

The rate of biodegradation of derivatized HA (e.g., the rate of release of derivatized HA) can be controlled, in part, by the degree of crosslinking of HA, and the quantity of the crosslinked HA loaded on the support. The residence time of unmodified HA in the human body is generally less than a week. However, when HA is derivatized, the residence time is appreciably increased. In general, an increase in the degree of crosslinking results in an increase in the time of residence. By controlling the degree of crosslinking, a crosslinked HA of desired residence time can be synthesized. In some embodiments, the derivatized HA selected for a particular use may have a biodegradation rate which is faster than the biodegradation rate of the support. The support, in fact, can be itself made of a sample of crosslinked HA having a slower rate of biodegradation than that of the derivatized HA loaded on the support.

The rate at which the gel, film or sponge of the HA derivative degrades and diffuses also depends on the insolubility, the density, and the degree of crosslinking of the modified HA in the composite. Just as gels, films and sponges which have a high degree of crosslinking are slow to degrade, modified HA which is more insoluble, or which has a higher degree of crosslinking, will degrade at a slower rate. Preferably, the density of modified HA in the film or sponge will be in the range of from about 0.1 mg/cm² to about 100 mg/cm². Those skilled in the art will know, or will be able to ascertain with no more than routine experimentation, the appropriate combination of insolubility, density and crosslinking that will yield a gel, film or sponge having the desired rate of degradation for a given situation.

The rate at which the cellular growth factor or cellular growth differentiation factor in the composites of the invention is released can also be controlled by varying the physical characteristics of the composite, such as porosity and interconnectivity, or varying the HA crosslink density. Typically, the retention of the cellular growth factor or cellular differentiation factor will range from about 1 day to 6 months, and preferably in the 2-4 weeks range.

A composite of the invention includes at least one member of the group consisting of a cell, a cellular growth factor and a cellular differentiation factor. The cell, cellular growth factor or cellular differentiation factor is impregnated in, or coupled to, the HA derivative or the HA derivative and biocompatible, biodegradable support. Suitable examples of the cell, cellular growth factor and cellular differentiation factor include mesenchymal stem cells from various tissue sources (e.g., cartilage, periosteum, synovium, bone marrow, fat, etc.), fibrochondrocytes, osteochondrocytes, chondrocytes, TGFβ supergene family members, such as BMPs, IGF, PDGF, GDFs, CDMPs and GFG, and tissue growth hormones. In addition, genes encoding for these proteins, as well as synthetic peptide analogues of these proteins can be contemplated. In a preferred embodiment, the composite include at least one member selected from the group consisting of mesenchymal stem cells, fibrochondrocytes, osteochondrocytes and chondrocytes. In another preferred embodiment, the composite includes both a cell and a cellular growth factor or cellular differentiation factor. The cellular growth and differentiation factors can provide molecular cuing for the cell, for example, to produce a meniscal tissue, or provide signals for the cell to differentiate down the meniscal tissue.

Typically, the cells, cellular growth factors and cellular differentiation factors are harvested or obtained from various sources and impregnated in, or coupled to, the HA derivative by methods known in the art. For example, autologous cells (e.g., chondrocytes or marrow-derived pluripotent stem cells) can be harvested, optionally expanded in culture, and loaded on to the meniscal implants by conventional cell seeding methods. The cell-loaded implants may be further cultured prior to implantation. Additionally, cellular growth factors or cellular differentiation factors can be loaded prior to implantation by various means. These factors can be loaded before (e.g., suspension, covalent linking, etc) or after (e.g., soak-loading, surface immobilization, etc.) manufacture of the implant.

The composite of the invention described above can be used for the meniscal augmentation device having a shape of a meniscal defect in the meniscus. The meniscal augmentation device includes at least one member of the group consisting of fibrochondrocytes, mesencymal stem cells and cellualr growth and differentiation factors, which is impregnated in, or coupled to, a biocompatible, biodegradable support and an HA derivative, as described above. The features, including preferred parameters, of the biocompatible, biodegradable support and HA derivative are as described above. Preferably, the cell, cellular growth factor or cellular differentiation factor includes at least one member selected from the group consisting of fibrochondrocytes, osteochondrocytes, periostealcell, synoviocytes, chondrocytes, mesencymal stem cells, cellular growth and differentiation factors, gene encoding for these proteins and synthetic peptide analogues of these proteins.

In one embodiment, the meniscal augmentation device is fabricated in the shape of the meniscal defect in the meniscus. Alternatively, the meniscal augmentation device is in the form of a sheet or film that can be cut, trimmed and contoured to fill the meniscal defect in the meniscus.

Another aspect of the invention is a method of regenerating or stimulating regeneration of meniscus tissues in a subject by the use of the composite and meniscal augmentation device of the invention described above. The method includes implanting the composite or the meniscal augmentation device at a treatment site, such as a site of the meniscus defect. Typically, the implanting can be done by surgical or arthroscopic insertion.

The implanted composition or meniscal augmentation device is located at a treatment site for an extended period of time (e.g., a time period of at least days, a week, a month, two months, six months, a year or longer than two years).

As used herein, the term “treating” refers to resulting in a beneficial clinical outcome of or exerts a positive influence on, the condition being treated with the composite of the invention compared with the absence of treatment. As used herein, a “treatment site” is the site in a subject that is in need of treatment for an articular defect, such as meniscal defect in the meniscus. The treatment site also includes the site in need of regeneration of meniscal tissues in a subject.

As used herein a subject is a mammal, preferably a human, but can also be an animal in need of veterinary treatment, such as a companion animal (e.g., dogs, cats, and the like), a farm animal (e.g., cows, sheep, pigs, horses, and the like) or a laboratory animal (e.g., rats, mice, guinea pigs, and the like).

The implantable composite and meniscal augmentation device of the invention can provide, for example, scaffolds that have tear strength and tear propagation resistance and can be surgically sutured or anchored to stabilize them within the meniscus defect so that they do not move during the healing and tissue regeneration. Once sutured in place, these scaffolds will provide a matrix into which the impregnated or coupled cells, or surrounding cells from, for example, the cartilage, bone and synovium, can begin to migrate and/or multiply, or cellular growth or differentiation factors can exert local activity. The HA derivative in the composite can influence on cell infiltration, the formation and degradation of a fibrin matrix, swelling of the matrix, phagocytosis and vascularisation. As the meniscal tissue regeneration takes place, the composite will be absorbed by the body and the regenerated tissue will restore function, reduce pain, and possibly retard or suspend the degenerative process caused by meniscus defects, e.g., loss of meniscal tissues.

### EXEMPLIFICATION

### Example 1

This example illustrates an embodiment of the invention in which a biscarbodiimide, p-phenylene-bis(ethylcarbodiimide), and HA are reacted at a molar equivalent ratio of 16.0% .

A solution of HA (5.4 mg/ml; 200-ml; 2.69 mequiv) was reacted with a solution of p-phenylene-bis(ethylcarbodiimide) (1 mg/ml in acetone; 46.1-ml; 0.215 mmol; 0.43 mequiv) according to a procedure described in U.S. Pat. Nos. 5,356,883, 5,502,081 and 6,013,679, the teachings of which are incorporated herein by reference in their entirety. The precipitate of the crosslinked HA was separated from the solution, washed, and resuspended in saline. The suspension was stirred for 2 days in a cold room to form a water-insoluble gel of about 4 mg/ml concentration. Chloroform equal to ½ of the volume of the aqueous solution was added to the solution and contents were vigorously stirred for seven days in the cold room. The reaction mixture was then centrifuged at 4° C. and 43 k rpm for one hour to remove chloroform. The aqueous/gel layer was aseptically collected and the concentration of sodium chloride in the collected aqueous/gel was adjusted to 1M. The mixture was stirred for 15 minutes under aseptic conditions. Ethanol equal to 3 volumes of the solution was added to precipitate the crosslinked HA and the precipitate was collected, squeezed to remove ethanol, and shredded into small pieces under aseptic conditions. The precipitate was re-dissolved in injection grade water to reconstitute a gel of desired concentration.

### Example 2

This example illustrates an embodiment of the invention in which a biscarbodiimide, p-phenylene-bis(ethylcarbodiimide), and HA are reacted at a molar equivalent ratio of 8.0%.

A solution of HA (5.4 mg/ml; 200-ml; 2.69 mequiv) was reacted with a solution of p-phenylene-bis(ethylcarbodiimide) (1 mg/ml in acetone; 23.0-ml; 0.108 mmol; 0.216 mequiv) according to a procedure described in U.S. Pat. Nos. 5,356,883, 5,502,081 and 6,013,679, the teachings of which are incorporated herein by reference in their entirety. The precipitate of the crosslinked HA was separated from the solution, washed, and resuspended in saline. The suspension was stirred for 2 days in a cold room to form a water-insoluble gel of about 4 mg/ml concentration. Chloroform equal to ½ of the volume of the aqueous solution was added to the solution and contents were vigorously stirred for seven days in the cold room. The reaction mixture was then centrifuged at 420 C. and 43 k rpm for one hour to remove chloroform. The aqueous/gel layer was aseptically collected and the concentration of sodium chloride in the collected aqueous/gel was adjusted to 1M. The mixture was stirred for 15 minutes under aseptic conditions. Ethanol equal to 3 volumes of the solution was added to precipitate the crosslinked HA and the precipitate was collected, squeezed to remove ethanol, and shredded into small pieces under aseptic conditions. The precipitate was re-dissolved in injection grade water to reconstitute a gel of desired concentration.

### Example 3

This example illustrates an embodiment of the invention in which a biscarbodiimide, p-phenylene-bis(ethylcarbodiimide), and HA are reacted at a molar equivalent ratio of 8.0% in MES buffer.

A solution of HA (15.0 mg/ml; 133.3-ml; 4.99 mequiv) in MES buffer (pH 5.5) was reacted with a solution of p-phenylene-bis(ethylcarbodiimide) (15 mg/ml in acetone; 2.8-ml; 0.2 mmol; 0.4 mequiv) according to a procedure described in U.S. Patent Application 2005/0136122 A1. The reaction mixture was thoroughly mixed (mixing with either a glass rod or an overhead mechanical stirrer, e.g., for about 1 minute, results in a white paste from the clear reaction mixture), and the mixture was allowed to stand at room temperature for about 96 hours. Sodium chloride (6.5 g, to make the mixture 5% by weight of sodium chloride) was mixed into the resulting gel, which was allowed to stand for 1 hour. The crosslinked HA gel was precipitated by addition into about 1.2 L of vigorously stirred ethanol. The precipitate was collected and dried under reduced pressure yielding the crosslinked hyaluronic acid. The dry crosslinked HA precipitate was milled. The powder was packed in a Tyvek®/Mylar® pouch, sealed and sterilized by ethylene oxide. The precipitate was re-dissolved in injection grade water to reconstitute a gel of desired concentration.

### Example 4

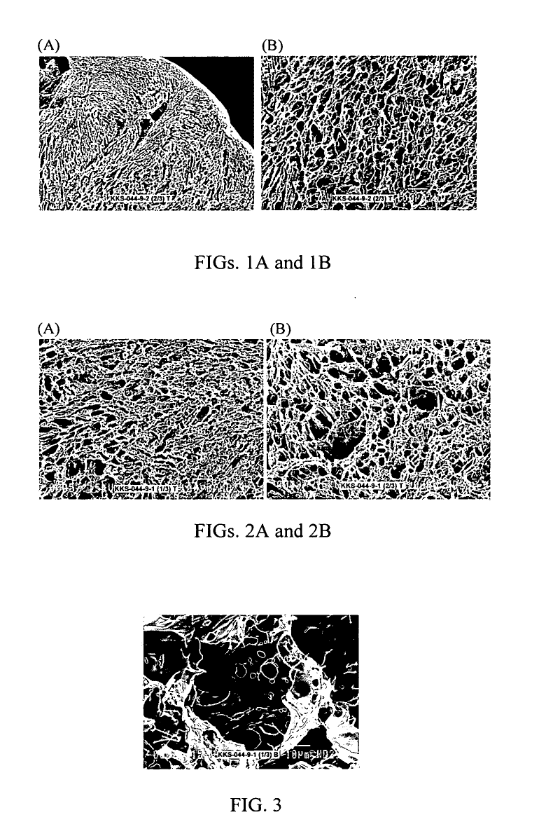

Example 4 describes the preparation of Sponge 1 shown in FIGS. 1A and 1B, an embodiment of the invention which is a composite including crosslinked HA derivative only. To make Sponge 1, a gel of crosslinked HA prepared according to the procedure described in Example 1, was poured into an 8 cm×8 cm mold under aseptic conditions. The mold containing the crosslinked HA gel was frozen at −45° C. and then freeze-dried under aseptic conditions for 24 hours under vacuum of less then 10 millimeters. The freeze-dried sponge was cut under aseptic conditions into 4 cm×4 cm pieces. These sponges were put in sterile pouches and sealed to keep them sterile.

### Example 5

Example 5 describes the preparation of Sponge 2 shown in FIGS. 2A and 2B, an embodiment of the invention which is a composite including crosslinked HA derivative only. To make Sponge 2, a gel of crosslinked HA prepared according to the procedure described in Examples 2 and 3, was poured into an 8 cm×8 cm mold under aseptic conditions. The mold containing the crosslinked HA gel was frozen at −45° C. and then freeze-dried under aseptic conditions for 24 hours under vacuum of less then 10 millimeters. The freeze-dried sponge was cut under aseptic conditions into 4 cm×4 cm pieces. These sponges were put in sterile pouches and sealed to keep them sterile.

### Example 6

This example describes the preparation of an embodiment of the invention, Sponge 3, a composite having HA derivative on both sides of a support made of collagen. The HA derivative has at least about 1% crosslinking, and was prepared according to the following procedure.

A solution of hyaluronic acid (MWt. 2.35×10⁶ Daltons, 1922 ml, 6 mg/ml, pH 4.75, 28.76 mmoles) in saline was crosslinked using a solution of cross-linker p-phenylene-bis(ethylcarbodiimide) in acetone (1 mg/ml, 246 ml, 1.15 mmoles). The crosslinked HA was precipitated, separated from the solution and washed with ethanol. A weighed portion of the precipitate was dissolved in sterile water to form crosslinked HA gel of about 7.7 mg/ml concentration.

Non-sterile collagen sponge was cut in to square pieces of desired dimensions. Crosslinked HA gel (7.7 mg/ml, 24 ml), prepared according to the procedure described above was poured in to the lower chamber of a 12 cm×8 cm mold and spread into a layer of even thickness. A 14 cm×10 cm piece of collagen sponge was placed on the top of the spread gel and it was covered with another layer of crosslinked HA gel (7.7 mg/ml, 24 ml). The collagen sponge was allowed to soak in the gel for 1 hour under aseptic condition in a refrigerator. The mold containing the composite was frozen at −46° C. and then freeze-dried for 24 hours under vacuum of less then 10 millimeters. The sides of the freeze-dried composite were trimmed to make a sponge, 11.5 cm×7.5 cm. This larger piece of sponge was then cut in to four 5.5 cm×3.5 cm pieces. Each piece was individually packed a Tyvek®/Mylar® pouch, sealed and sterilized by ethylene oxide (EtO).

### Examples 7-9 (prophetic)

In each of these examples, reagents are used in the amounts indicated in Table 1. Uncrosslinked HA (2.0 g) is dissolved in 133.4 mL of MES buffer at the pH 5.5 and combined with a 15 mg/mL acetone solution of p-phenylene-bis(ethylcarbodiimide) (PBCDI), resulting in the specified molar equivalent ratio (MER %) and mol % between PBCDI:HA. The reaction mixture is then thoroughly mixed (mixing with either a glass rod or an overhead mechanical stirrer, e.g., for about 1 minute, can result in a white paste from the clear reaction mixture), and the mixture is poured in to the molds designed in the shape of the meniscus or any other desired shape, allowed to stand at room temperature for about 72 hours. The mold containing the crosslinked HA gel is frozen at −45° C. and then freeze-dried for 24 hours under vacuum of less then 10 millimeters. The freeze dried material is soaked and washed with organic solvent to remove the undesired contaminants of the reaction. The solvent is removed and the scaffold was dried under vacuum. The dried scaffold is sealed in Tyvek/Mylar pouch and sterilized by EtO (ethylene oxide).

TABLE 1
Details for synthesizing crosslinked HA in Examples 7-9

| | PBCDI | | | | Hyaluronic acid | | | | | |
|---|---|---|---|---|---|---|---|---|---|---|
| | (15 mg/mL) | | | | (15 mg/mL) | | | | | |
| Example | mL | mg | mmol | mequiv | mL | g | mmol | mequiv | MER % | Mol % |
| 7 | 17.8 | 267 | 1.25 | 2.5 | 133.3 | 2.0 | 5.0 | 5.0 | 50 | 25 |
| 8 | 26.7 | 400 | 1.87 | 3.74 | 133.3 | 2.0 | 5.0 | 5.0 | 75 | 37.4 |
| 9 | 35.6 | 534 | 2.5 | 5.0 | 133.3 | 2.0 | 5.0 | 5.0 | 100 | 50 |

### Example 10 (prophetic)

Sponges 1 and 2 of Examples 4 and 5, the composite made by method of Example 6 and sponges that can be made by the methods described in Examples 7-9, respectively, can provide scaffolds adapted for the loading and ingrowth of cells (such as meniscal fibrochondrocytes), cellular growth factors and/or cellular differentiation factors. FIG. 3 shows a cross sectional SEM image of Sponge 2 showing interconnected pores that can provide cues for the loaded cells to move or migrate and multiply, or for the loaded cellular growth or differentiation factors to move and migrate and exert local activity, to thereby treating meniscal defects.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A composite for treating an articular defect, comprising:
a) a hyaluronic acid derivative, wherein carboxyl functionalities of the hyaluronic acid derivative are each independently derivatized to include an N-acylurea or O-acyl isourea, or both N-acylurea and O-acyl isourea; and
b) at least one member of the group consisting of a cell, a cellular growth factor and a cellular differentiation factor, which is impregnated in, or coupled to, the hyaluronic acid derivative.

2. The composite of claim 1, wherein the composite includes at least one member of the group consisting of mesenchymal stem cells, periosteal cells, synoviocytes, fibrochondrocytes, osteochondrocytes, chondrocytes, TGFβ supergene family members, tissue growth hormones, encoding genes thereof, and synthetic peptide analogues thereof.

3. The composite of claim 1, wherein at least 1% by mole of the carboxyl functionalities have been derivatized.

4. The composite of claim 3, wherein at least 25% by mole of the derivatized carboxyl functionalities are O-acyl isoureas and/or N-acylureas.

5. The composite of claim 1, wherein the hyaluronic acid derivative includes at least one crosslink represented by the following structural formula:
HA′-U—R₂—U—-HA′
wherein:
each HA′ is the same or a different hyaluronic acid molecule;
each U is independently an optionally substituted O-acyl isourea or N-acyl urea; and
each R₂ is independently a substituted or unsubstituted hydrocarbylene group optionally interrupted by one or more heteroatoms.

6. The composite of claim 1, wherein the composite is an implantable composite.

7. The composite of claim 6, wherein the composite has interconnected pores of sizes that can provide molecular cuing and scaffolding for the impregnated or coupled cell, or path for migration of the cellular growth factor or cellular differentiation factor.

8. The composite of claim 6, wherein the composite is in a form selected from the group consisting of sponge, film, sheet, gel, thread, tube, woven and non-woven fabrics, cords and meshes.

9. The composite of claim 8, wherein the composite is in the form of a sponge.

10. The composite of claim 9, wherein the composite is in the form of a sheet or film.

11. The composite of claim 8, wherein the sheet or film is hydrophilic.

12. The composite of claim 1 1, wherein the sheet or film can be stacked together for surgical suturing or anchoring.

13. The composite of claim 7, wherein the hyaluronic acid derivative is in a form selected from the group consisting of sponge, gel, sheet and film.

14. The composite of claim 1, further including a biocompatible, biodegradable support, wherein the hyaluronic acid derivative is at the support.

15. The composite of claim 14, wherein the support includes at least one member selected from the group consisting of crosslinked alginates, gelatin, collagen, crosslinked collagen, collagen derivatives, crosslinked hyaluronic acid, chitosan, chitosan derivatives, cellulose and derivatives thereof, dextran derivatives, polyanionic polysaccharides and derivatives thereof, polylactic acid (PLA), polyglycolic acid (PGA), a copolymer of a polylactic acid and a polyglycolic acid (PLGA), lactides, glycolides, polyoxanones, polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of polyamino acids with polyethylene glycol, polypeptides, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone and polyvinylalcohol.

16. The composite of claim 1, further including a material that enhances adherence of the composite to tissue.

17. The composite of claim 16, wherein the material that enhances adherence of the composite to tissue is a polymer selected from the group consisting of fibrin, collagen, crosslinked collagen, collagen derivative and a polymer that includes a peptide sequence having arginine, glycine and aspartic acid.

18. A meniscal augmentation device having a shape of a meniscal defect in meniscus, comprising:
a) a hyaluronic acid derivative, wherein carboxyl functionalities of the hyaluronic acid derivative are each independently derivatized to include an N-acylurea or O-acyl isourea, or both N-acylurea and O-acyl isourea; and
b) at least one member of the group consisting of a cell, a cellular growth factor and a cellular differentiation factor, which is impregnated in, or coupled to, the hyaluronic acid derivative.

19. The meniscal augmentation device of claim 18, wherein said at least one cell, cellular growth factor or cellular differentiation factor includes at least one member selected from the group consisting of mesenchymal stem cells, periosteal cells, synoviocytes, fibrochondrocytes, osteochondrocytes, chondrocytes, TGFβ supergene family members, tissue growth hormones, encoding genes thereof, and synthetic peptide analogues thereof.

20. The meniscal augmentation device of claim 18, wherein the device includes a meniscus fibrochondrocyte.

21. The meniscal augmentation device of claim 18, wherein at least 1% by mole of the carboxyl functionalities have been derivatized.

22. The meniscal augmentation device of claim 21, wherein at least 25% by mole of the derivatized carboxyl functionalities are O-acyl isoureas and/or N-acylureas.

23. The meniscal augmentation device of claim 18, wherein the hyaluronic acid derivative includes at least one crosslink represented by the following structural formula:
HA′-U—R₂—U—HA′
wherein:
each HA′ is the same or a different hyaluronic acid molecule;
each U is independently an optionally substituted O-acyl isourea or N-acyl urea; and
each R₂ is independently a substituted or unsubstituted hydrocarbylene group optionally interrupted by one or more heteroatoms.

24. The meniscal augmentation device of claim 18, wherein the composite has interconnected pores of sizes that can provide molecular cuing for the impregnated or coupled cell, or a path for migration of the cellular growth factor or cellular differentiation factor.

25. The meniscal augmentation device of claim 18, further including a biocompatible, biodegradable support, wherein the hyaluronic acid derivative is at the support.

26. The meniscal augmentation device of claim 18, wherein the support include at least one member selected from the group consisting of crosslinked alginates, gelatin, collagen, crosslinked collagen, collagen derivatives, crosslinked hyaluronic acid, chitosan, chitosan derivatives, cellulose and derivatives thereof, dextran derivatives, polyanionic polysaccharides and derivatives thereof, polylactic acid (PLA), polyglycolic acid (PGA), a copolymer of a polylactic acid and a polyglycolic acid (PLGA), lactides, glycolides, polyoxanones, polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of polyamino acids with polyethylene glycol, polypeptides, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone and polyvinylalcohol.

27. The meniscal augmentation device of claim 18, further including a material that enhances adherence of the composite to tissue.

28. The meniscal augmentation device of claim 27, wherein the material that enhances adherence of the composite to tissue is a polymer selected from the group consisting of fibrin, collagen, crosslinked collagen, collagen derivative and a polymer that includes a peptide sequence having arginine, glycine and aspartic acid.

29. The meniscal augmentation device of claim 18, wherein the device is fabricated in the shape of the meniscal defect in the meniscus.

30. The meniscal augmentation device of claim 18, wherein the device is in the form of a sheet or film that can be cut, trimmed and contoured to fill the meniscal defect in the meniscus.

31. A method of regenerating or stimulating regeneration of meniscus tissues in a subject, comprising implanting a composite at a meniscus defect site, the composite including:
a) a hyaluronic acid derivative, wherein carboxyl functionalities of the hyaluronic acid derivative are each independently derivatized to include an N-acylurea or O-acyl isourea, or both N-acylurea and O-acyl isourea; and
b) at least one member of the group consisting of a cell, a cellular growth factor and a cellular differentiation factor, which is impregnated in, or coupled to, the hyaluronic acid derivative.

32. The method of claim 31, wherein the cell, and the cellular growth and differentiation factors include at least one member selected from the group consisting of mesenchymal stem cells, periosteal cells, synoviocytes, fibrochondrocytes, osteochondrocytes, chondrocytes, TGFβ supergene family members, tissue growth hormones, encoding genes thereof, and synthetic peptide analogues thereof.

33. The method of claim 32, wherein the composite include a meniscus fibrochondrocyte.

34. The method of claim 31, further including the step of stabilizing the composite within the meniscus defect so that the composite does not move during the regenerating or stimulating regeneration of meniscus tissues.

35. The method of claim 31, wherein at least 1% by mole of the carboxyl functionalities have been derivatized.

36. The method of claim 31, wherein at least 25% by mole of the derivatized carboxyl functionalities are O-acyl isoureas and/or N-acylureas.

37. The method of claim 31, wherein the hyaluronic acid derivative includes at least one crosslink represented by the following structural formula:
HA′-U—R₂—U—HA′
wherein:
each HA′ is the same or a different hyaluronic acid molecule;
each U is independently an optionally substituted O-acyl isourea or N-acyl urea; and
each R₂ is independently a substituted or unsubstituted hydrocarbylene group optionally interrupted by one or more heteroatoms.

38. The method of claim 31, wherein the composite has interconnected pores of sizes that can provide molecular cuing and scaffolding for the impregnated or coupled cell to migrate through, or a path for migration of the impregnated or coupled cellular growth factor or cellular differentiation factor.

39. The method of claim 31, wherein the composite further includes a biocompatible, biodegradable support, the hyaluronic acid derivative being at the support.

40. The method of claim 39, wherein the support includes at least one member selected from the group consisting of crosslinked alginates, gelatin, collagen, crosslinked collagen, collagen derivatives, crosslinked hyaluronic acid, chitosan, chitosan derivatives, cellulose and derivatives thereof, dextran derivatives, polyanionic polysaccharides and derivatives thereof, polylactic acid (PLA), polyglycolic acid (PGA), a copolymer of a polylactic acid and a polyglycolic acid (PLGA), lactides, glycolides, polyoxanones, polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of polyamino acids with polyethylene glycol, polypeptides, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone and polyvinylalcohol.

41. The method of claim 31, wherein the composite further includes a material that enhances adherence of the composite to tissue.

42. The method of claim 41, wherein the material that enhances adherence of the composite to tissue is a polymer selected from the group consisting of fibrin, collagen, crosslinked collagen, collagen derivative and a polymer that includes a peptide sequence having arginine, glycine and aspartic acid.

43. The method of claim 31, further including the step of fabricating the composite in the shape of the meniscal defect in the meniscus.

44. The method of claim 31, further including the steps of: forming the composite in a sheet or film; and cutting, trimming and contouring the sheet or film to fill the meniscal defect in the meniscus.

45. A composite for treating an articular defect, comprising:
a) a hyaluronic acid derivative, wherein the hyaluronic acid derivative is prepared by reacting an uncrosslinked hyaluronic acid with a biscarbodimide in the presence of a pH buffer in a range of between about 4 and about 8; and
b) at least one member of the group consisting of a cell, a cellular growth factor and a cellular differentiation factor, which is impregnated in, or coupled to, the hyaluronic acid derivative.

46. A meniscal augmentation device having a shape of a meniscal defect in meniscus, comprising:
a) a hyaluronic acid derivative, wherein the hyaluronic acid derivative is prepared by reacting an uncrosslinked hyaluronic acid with a biscarbodimide in the presence of a pH buffer in a range of between about 4 and about 8; and
b) at least one member of the group consisting of a cell, a cellular growth factor and a cellular differentiation factor, which is impregnated in, or coupled to, the hyaluronic acid derivative.

47. A method of regenerating or stimulating regeneration of meniscus tissues in a subject, comprising implanting a composite at a treatment site of the meniscus defect, the composite including:
a) a hyaluronic acid derivative, wherein the hyaluronic acid derivative is prepared by reacting an uncrosslinked hyaluronic acid with a biscarbodimide in the presence of a pH buffer in a range of between about 4 and about 8; and
b) at least one member of the group consisting of a cell, a cellular growth factor and a cellular differentiation factor, which is impregnated in, or coupled to, the hyaluronic acid derivative.
